(19) United States
(12) Patent Application Publication
(10) Pub. No.: US 2003/0059488 A1
(43) Pub. Date: Mar. 27, 2003

(54) Bioflavanoid-rich potent extract and process for the extraction from white oak bark and pine bark

(76) Inventor: Eugene Daniels, Campbellton, FL (US)

Correspondence Address: Lawrence L. Carnes, Innovation Park, 1736 W. Paul Dirac Dr., Tallahassee, FL, 32310, US

(21) Appl. No.: 10/207,729

(22) Filed: Jul. 29, 2002

Related U.S. Application Data
(60) Provisional application No. 60/308,546, filed on Jul. 30, 2001.

(51) Int. Cl. A61K 35/78

(52) U.S. Cl. 424/770; 424/771

(57) ABSTRACT

A bioflavanoid rich potent extract and process for the extraction from oak bark and pine bark that is highly enriched in proanthocyainds. The present invention provides for component liquids comprised of approximately 2% active ingredients of gallic acids and tannic acids which are then added to other inactive ingredients which include corn syrup and flavoring.

### BACKGROUND OF THE INVENTION

1. Field of the Invention

The present invention relates generally to a bioflavanoid-rich potent extract and process for the extraction from white oak bark and pine bark and more particularly to an extraction which will provide for a bioflavoniod-rich potent extract which is used to fight free radicals and maintain capillary health. The instant invention focuses on the extraction of proanthocyanidins from white oak bark and pine bark. White oak bark is one of the strongest, natural astringent herbs available, as well as providing anti-inflammatory and anti-septic properties. Pine bark is one of the strongest, natural antiseptic and astringent agents which acts to shrink mucous membranes and raw surfaces as well as dry up secretions.

2. Description of the Prior Art

Hot water extraction of proanthocyanidins from various biological materials is well known in the prior art. For example, U.S. Pat. No. 5,968,517 issued to Duncan et al wherein disclosed is a method for extraction and isolation of proanthocyanidins from biological material. The method involved includes the steps of hot water extraction of the material after comminution using deoxygenated water, separation of the solids from the liquor, concentration of liquor into a concentrated solution, and waste streams and drying the concentrated solution to a solid product.

Another example can be seen in U.S. Pat. No. 5,863,437 issued to Ulbrich et al. In this patent there is disclosed a method and device for separating a mixture having at least two components, wherein a separation phase is impacted by the mixture to be separated and then the separation phase loaded with the mixture is eluted with at least one fluid.

Yet another example is seen in U.S. Pat. No. 5,762,936 issued to Ronzio et al wherein disclosed is a preparation of extracts of seed coats of lentil extracted with a range of volatile solvents such as methanol, acetone, singly or a mixture with water, and food solvents to yield such extracts that are water-soluble, which contain a rich mixture of condensed tannins, together with a flabanone and flavonols and phenolacids and which possess the ability to quench organic free radicals, to scavenge superoxide.

Another example is seen in U.S. Pat. No. 6,066,327 issued to Gubernick et al wherein disclosed is a cosmetic or pharmaceutical composition for topical application to the skin.

A final example can be seen in U.S. Pat. No. 5,238,680 issued to Connolly. This patent provides a method for extracting tannins from bark and multi-stage apparatus suitable therefore.

Accordingly, there exists a need to provide for a bioflavanoid-rich potent extract and a process for the extraction from white oak bark and pine bark which is used to fight free radicals and maintain capillary health. No previous efforts have been disclosed which provide the benefits intended with the present invention. Additionally, prior techniques do not suggest the present inventive combination of component elements as disclosed and claimed herein. The present invention achieves its intended purposes objectives and advantages over the prior art device through a new, useful and unobvious combination of component elements, which is simple to use, with the utilization of a minimum number of functioning ingredients, at a reasonable cost to manufacture, test and by employing only readily available materials.

### SUMMARY OF THE INVENTION

The present invention provides for a bioflavonoid-rich potent extract and process for the extraction from white oak bark and pine bark which is adapted to be used to fight free radicals and maintain capillary health. The composition of the present invention provides for a potent extract which can be used for its free radical fighting capabilities and for a variety of conditions related to permeability as well as capillary health. The present invention is indicated for a variety of health related conditions including being used as an antiseptic and as an astrengic agent which shrinks mucous membranes, raw surfaces, and to dry up secretions. In combination, the white oak bark and pine bark extract acids excel in relieving cold symptoms very effectively and at a rapid rate. Also, the present invention provides relief for poor distribution of microcirculatory blood flow in the brain and heart, as well as altered capillary fragility and permeability in diabetes mellitus. In addition, the instant invention also is indicated for chronic arterial/venous insufficiency in the extremities and for altered platelet aggregation and other characteristics of blood flow in capillaries. The present invention is also indicated for a break down in the elastic fibers of the collegian and elastain capillaries due to its free radical and enzyme action. It is also indicated for microangiopathy of the retina, enema of the lymph nodes, varicous veins and other symptoms of the problems listed above. The instant invention is also indicated for the cumulative effects of aging and also reduces the risks of degenerative diseases.

The present invention provides a method for an extraction and isolation of tannic and gallic acid from botanical material. This invention focuses on the extraction of tannic and gallic acid from the bark of trees, oak and pine respectively.

It has been determined that by preparing an extraction of proanthocianids extracts from white oak bark and pine bark, a composition of bioflavanoid rich extract can be produced which will fight free radicals and maintain capillary health.

The present invention provides a method for extraction and isolation of tannic and gallic acid from botanical material. This invention focuses on the extraction of tannic and gallic acid from the bark of oak and pine trees. Once the bark is selected, it must be screened in order to prevent the use of a contaminated source. The screening removes stones, rocks and additional extraneous matter. After the bark has been screened, it is washed and submersed into boiling water. The oak bark is heated within one vessel and the pine bark is heated within another vessel, with vacuum filtration removing the components from each of the vessels. In addition, vessels 3 & 4 are combined within vessel 5, which contains yields of 2% active ingredients of gallic and tannic acids for mixture with other inactive ingredients.

Therefore, it is an object of this invention to obtain a bioflavanoid rich potent extract that is highly enriched in proanthocyainds.

It is a further object of this invention to provide for a process which will result in a bioflavanoid rich potent extract which is used to fight free radicals and maintain capillary health.

Still another object of the present invention is to provide for a potent extract which can be used as an antiseptic and an astringent agent that will shrink mucous membranes and raw surfaces and dry up secretions.

Yet another object of the present invention is to provide for a combination of potent extract acids from white oak bark and pine bark that excel in relieving cold symptoms very effectively and at a rapid rate.

Although there have been other inventions related to bioflavanoid-rich extracts, none of the previous inventions provide the benefits of the present invention which is to provide potent anti-inflammatory and anti-septic properties which will act as a natural antiseptic and astrengent agent in order to shrink mucous membranes and

The foregoing has outlined some of the more pertinent objects of the present invention. These objects should be construed to be merely illustrative of some of the more prominent features of the application of the intended invention. Many other beneficial results can be obtained by applying the disclosed invention in a different manner or modifying the invention within the scope of the disclosure. Accordingly, a fuller understanding of the invention may be had by referring to the detailed description of the preferred embodiments in addition to the scope of the invention taken in conjunction with the accompanying drawings.

### BRIEF DESCRIPTION OF THE DRAWINGS

FIG. 1 is a diagrammatic representation of a batch process of the instant invention.

### DESCRIPTION OF THE PREFERRED EMBODIMENT

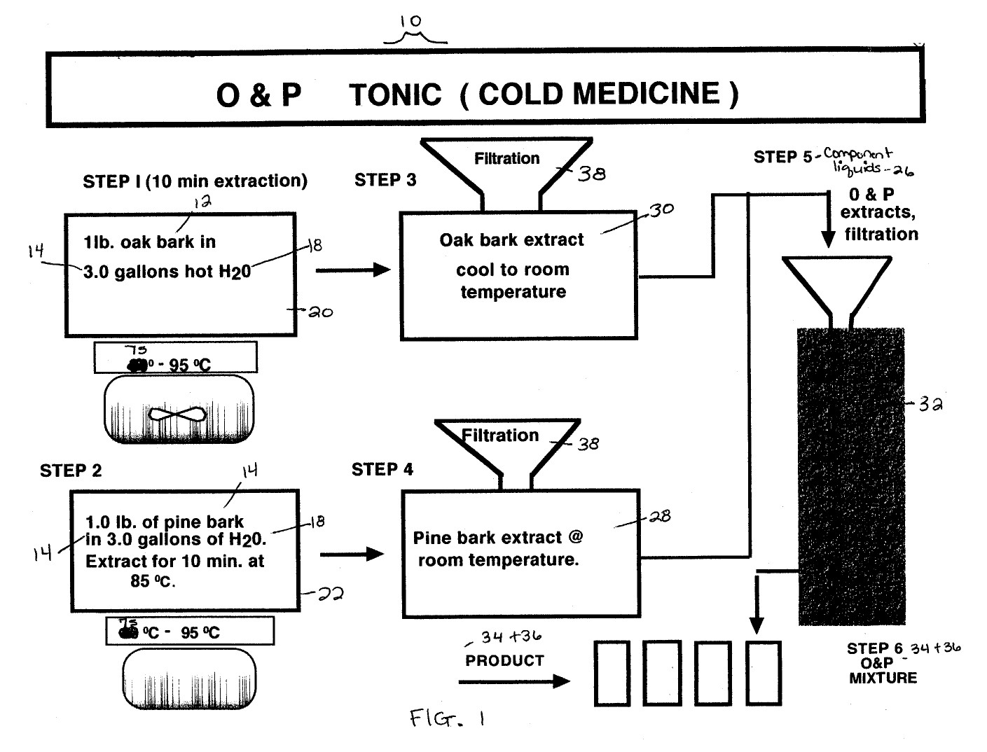

Referring to FIG. 1, the present invention provides a method for an extraction and isolation of tanic and gallic acid from the bark 10 of oak and pine trees respectively. The oak bark 12 and pine tree bark 14 is carefully screened in order to prevent any contaminated sources in the present invention. The screening process is designed to remove stones, rocks and other extraneous matter. The bark 10 of the instant invention may be fresh, wet or dry, and may be comprised of inner or outer bark 10 from oak and pine trees. After the above screening process is finalized, the bark 10 is selected and is thoroughly washed at room temperature. The bark is then added to previously boiled water in a reaction vessel. The preferred extraction during this process for the present invention may occur with or without agitation. Preferably, the process provides for stirring the preferred extraction in order to recover the end product at a faster rate.

The preferred embodiment extraction process in step 1 provides for a resultant stream of one pound of bark 10 which is added to previously to three gallons of boiled water 18 from 75-95 degrees Celsius. Thus the preferred extraction process is a batch operation either with or without agitation. Therefore, the oak bark 12 is heated in a batch operation in vessel one 20 while in step two, the pine bark 14 is heated in vessel two 22.

Hence, step two provides for a resultant stream of one pound of pine bark 14 which is added to three gallons of boiled water 24 from 75-95 degrees Celsius. The oak bark 12 and pine bark 14 are then boiled for twenty minutes to six hours. In the preferred embodiment, it has been found that two hours of boiling at atmospheric pressure is optimal. The present invention provides alternatively for a series of continuous stirred reactors, or a plug flow continuous reactor may be used to accomplish the hot water extraction. Thus, a major advantage of the batch process is the variability of time intervals desired between stages once the bark solids are removed from the hot water 24.

In several embodiments of the present invention where continuous extraction in a series of one or more stirred reactors or plug flow continuous reactors occurs it has been found that the extraction time can be reduced to the ten minute range or the like.

In step three, the present invention provides for the oak bark extract to be removed from the boiling water vessels by vacuum filtration as many times as desired including in practice normally twice.

In step four, the present invention provides for the removal of the pine bark 14 from the boiling water vessel by vacuum filtration 38 as many times as desired including in practice normally twice.

In step five, the present invention provides for the combination of the component liquids 26 of pine bark extract 28 and oak bark extract 30 into a vessel 32 at room temperature. This vessel 32 of the present invention now provides component liquids 26 comprised of 2% active ingredients of gallic acids 34 and tannic acids 36. These active gallic 34 and tannic 36 compounds are then added in step six to other inactive ingredients which include corn syrup and flavor.

In other embodiments of the present invention, the percentage of active ingredients of gallic acids 34 and tannic acids 36 may provide yields between 0.5 to 10% by weight of proanthocyanidins based on dried bark weight.

While the invention has been particularly shown and described with reference to an embodiment thereof, it will be understood by those skilled in the art that various changes in form and detail may be made without departing from the spirit and scope of the invention.

I claim:

1. A bioflavanoid rich potent extract, wherein said extract contains yields in the range of about 2 % active ingredients of gallic and tannic acids for mixture with other inactive ingredients.

2. A bioflavanoid rich potent extract, as in claim 1 wherein said extract has a percentage of active ingredients of gallic acids and tannic acids which may provide yields between 0.5 to 10% by weight of proanthocyanidins based on dry bark weight.

3. A method for extraction and isolation of tannic and gallic acid from the bark of oak and pine trees respectively; said method comprising the steps of:
a) carefully screening the oak bark and pine tree bark which may be fresh, wet or dry, and may be comprised of inner or outer said bark;
b) said bark is selected and thoroughly washed at room temperature;
c) said bark is then added to previously boiled water in a reaction vessel, the preferred extraction during this process may occur with or without agitation, preferably, the process provides for stirring said preferred extraction in order to recover the end product at a faster rate;
d) said extraction process provides for a resultant stream of one pound of said bark which has been previously added to three gallons of boiled water in a range of 75-95 degrees Celsius, thus said extraction process is a batch operation either with or without agitation;
e) said oak bark is heated in said batch operation in vessel one and said pine bark in said batch operation is heated in vessel two, which provides during this step for a resultant stream of one pound of said pine bark which is added to three gallons of said boiled water in the range of about 75-95 degrees Celsius, said oak bark and said pine bark are then boiled in said water for anywhere from twenty minutes to six hours, preferably, it has been found that two hours of boiling said mixture in said water at atmospheric pressure is optimal, alternatively, a series of continuous stirred reactors, or a plug flow continuous reactor may be used to accomplish the hot water extraction;
f) said oak bark extract is removed from said boiling water vessels by vacuum filtration as many times as desired, including in practice, normally twice
g) said pine bark is removed from said boiling water vessel by said vacuum filtration as many times as desired including in practice, normally twice;
h) combination of the component liquids of said pine bark extract and said oak bark extract into a vessel at room temperature results in the said component liquids being comprised of 2% active ingredients of gallic acids and tannic acid;
i) said active gallic and said active tannic compounds are then added to other inactive ingredients, which include corn syrup and flavor.

4. The method according to claim 3, wherein the percentage of active ingredients of said gallic acids and said tannic acids may provide yields between 0.5 to 10% by weight of proanthocyanidins based on said dried bark weight.
